(19) United States
(12) Patent Application Publication
(10) Pub. No.: US 2004/0067268 A1
(43) Pub. Date: Apr. 8, 2004

(54) Henna product

(76) Inventors: Mark Constantine, Dorset (GB); Margaret Constantine, Dorset (GB); Helen Ambrosen, Dorset (GB)

Correspondence Address: Westman Champlin & Kelly, International Centre Suite 1600, 900 Second Avenue South, Minneapolis, MN, 55402-3319, US

(21) Appl. No.: 10/450,598

(22) PCT Filed: Dec. 13, 2001
(86) PCT No.: PCT/GB01/05525

(30) Foreign Application Priority Data
Dec. 15, 2000 (GB) 0030705.8

(51) Int. Cl. A61K 7/06; A61K 35/78

(52) U.S. Cl. 424/757; 424/74

(57) ABSTRACT

A product comprising as its main components henna and cocoa butter with the product having a solid form. Also a method of forming a solid form product comprising the step of mixing henna and cocoa butter to form a paste and allowing the paste to harden. Additives may be provided, such as a surfactant, a conditioning agent, a fragrance, a herb, a fruit extract, a vegetable, a natural way or butter, a synthetic wax, a humectant, glycerine, a vegetable oil. Preferably, the product is form of 40% to 90% henna by weight and 10% to 60% cocoa butter by weight. More preferably the product is formed of 60% to 80% henna by weight and 20% to 40% cocoa butter by weight. Most preferably the product is formed of 70% henna by weight and 30% cocoa butter by weight.

The present invention relates to products, preferably cosmetic products, used for dyeing. Although the invention is primarily for use on hair it may also be used on skin and other materials.

Henna has traditionally been sold as a powder and occasionally as a paste, and as a colouring in hair rinses, fabric dyes, etc.

Hair treatments and products, particularly cosmetic products, used for dyeing are conventionally supplied in containers made of a rigid or semi-rigid plastic material or of foil in the form of sachets. The container adds significantly to the costs of the product and environmental pollution is caused by disposal of the containers. Henna has been sold in containers of the aforementioned type.

According to a first aspect of the present invention there is provided a product comprising as its main components henna and cocoa butter with the product having a solid form.

According to a second aspect of the present invention there is provided a method of forming a solid form product comprising the step of mixing henna and cocoa butter to form a paste and allowing the paste to harden.

Preferably the product is formed of 40% to 90% henna by weight and 10% to 60% cocoa butter by weight. More preferably the product is formed of 60% to 80% henna by weight and 20% to 40% cocoa butter by weight. Most preferably the product is formed of 70% henna by weight and 30% cocoa butter by weight.

Additives may be provided, such as a surfactant, a conditioning agent, a fragrance, a herb, a fruit extract, a vegetable, a natural wax or butter, a synthetic wax, a humectant, glycerine, a vegetable oil.

Embodiments of the present invention will now be described in more detail by way of further example only.

In accordance with the present invention it has been determined that the simple mixture of henna and cocoa butter produces a very stable solid form product. The product can be very easily formed into many useful and/or novelty shapes. These can be displayed to form an attractive feature of the product in a shop.

The solid form of the product according to the invention enables a beneficial reduction in costs and avoidance of environmental pollution to be achieved by avoiding the necessity of a container of the aforementioned type. Further, by virtue of being in solid form, the product can accept imprints during a moulding process.

Cocoa butter gives the product a good texture and, when the product is made into a paste form by admixture of water, it promotes easy application and it also conditions the hair.

Although the invention relates to dyeing and cosmetic products generally, a description will be given in relation to a hair dye.

One main component of the product is henna, which is a colouring agent and which adds shine to the hair. Herbs which can add colour to the hair, e.g. chamomile, can be added to the mixture and also herbs which condition only.

Cocoa butter is crucial to forming a stable solid product which promotes easy application and gives condition to the hair. An unacceptably substandard product would result if this component were wholly replaced; but other natural waxes, butters, synthetic waxes and the like can be used as partial replacements.

A mixture of 70% henna and 30% cocoa butter (by weight) forms a paste that hardens over a period of a few hours. The resultant solid material can be cut and small pieces can be broken off to suit specific requirements. The material and its easy application is thus in stark contrast to the usual form for a henna to be packaged in, this being a powder.

One example of the relative ratios of the components is given above. These can of course be varied, the requirements being only that a useable solid form product results. It appears most preferable for the henna to constitute 60%. to 80% of the initial mixture and cocoa butter 20% to 40%. Stated generally, the broad range is for the henna to constitute 40% to 90% and.the cocoa butter 10% to 60%.

Small amounts of additives can be included, such as surfactants, conditioning agents, fragrances, herbs, fruit extracts, vegetables and the like. Other beneficial additives can include other natural waxes or butters, synthetic waxes, surfactants, humectants, for example glycerine, herbs in powder or whole form, vegetable oils, and the like. Further, a colouring additive may be used to provide lettering or the like in the product.

Although the above description has been of a solid dyeing cosmetic product, it will be appreciated that the invention is equally well suited to a product suitable for dyeing many materials, for example, leather.

1. A product comprising as its main components henna and cocoa butter with the product having a solid form.

2. A product as claimed in claim 1 prepared from a mixture including 40% to 90% by weight of henna.

3. A product as claimed in claim 2 prepared from a mixture including 60% to 80% by weight of henna.

4. A product as claimed in any preceding claim prepared from a mixture including 10% to 60% by weight of cocoa butter.

5. A product as claimed in claim 4 prepared from a mixture including 20% to 40% by weight of cocoa butter.

6. A product as claimed in any preceding claim prepared from a mixture including 70% by weight of henna and 30% by weight of cocoa butter.

7. A product as claimed in any of claims 1 to 5 and further including one or more additives.

8. A product as claimed in claim 7 wherein the additive is one or more of a surfactant, a conditioning agent, a fragrance, a herb, a fruit extract, a vegetable.

9. A product as claimed in claim 7 wherein the additive is one or more of a natural wax or butter, a synthetic wax, a humectant, glycerine, a herb in powder or whole form, a vegetable oil.

10. A product as claimed in claim 7 including a fragrance as an additive.

11. A product as claimed in claim 7 including a fruit juice as an additive.

12. A. product as claimed in any preceding claim and further including a colouring additive providing lettering or the like in the product.

13. A product as claimed in any preceding claim wherein the product has a surface imprint.

14. A product as claimed in any preceding claim, wherein the product is a cosmetic product.

15. A method of forming a solid form product comprising the step of mixing henna and cocoa butter to form a paste and allowing the paste to harden.

16. A method as claimed in claim 15 comprising the step of moulding the mixture in a mould.
